(19) United States
(12) Patent Application Publication
(10) Pub. No.: US 2005/0214329 A1
(43) Pub. Date: Sep. 29, 2005

(54) Vaccine

(76) Inventors: Craig Anthony Joseph Laferriere, King Prussia, PA (US); Jan Poolman, Rixensart (BE)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/498,900

(22) PCT Filed: Dec. 18, 2002
(86) PCT No.: PCT/EP02/14476

§ 371 Date: Feb. 28, 2005

(30) Foreign Application Priority Data
Dec. 18, 2001 (GB) 0130215.7

(51) Int. Cl. A61K 39/09

(52) U.S. Cl. 424/244.1

(57) ABSTRACT

The present invention provides an optimal formulation of multiple serotype Streptococcus pneumoniae conjugate vaccines.

### FIELD OF THE INVENTION

The present invention relates to an improved Streptococcus pneumonia vaccine.

### BACKGROUND OF THE INVENTION

Children less than 2 years of age do not mount an immune response to most polysaccharide vaccines, so it has been necessary to render the polysaccharides immunogenic by chemical conjugation to a protein carrier. Coupling the polysaccharide, a T-independent antigen, to a protein, a T-dependent antigen, confers upon the polysaccharide the properties of T dependency including isotype switching, affinity maturation, and memory induction.

However, there can be issues with repeat administration of polysaccharide-protein conjugates, or the combination of polysaccharide-protein conjugates to form multivalent vaccines. For example, it has been reported that a Haemophilis influenzae type b polysaccharide (PRP) vaccine using tetanus toxoid (TT) as the protein carrier was tested in a dosage-range with simultaneous immunization with (free) TT and a pneumococcal polysaccharide-TT conjugate vaccine following a standard infant schedule. As the dosage of the pneumococcal vaccine was increased, the immune response to the PRP polysaccharide portion of the Hib conjugate vaccine was decreased, indicating immune interference of the polysaccharide, possibly via the use of the same carrier protein (Dagan et al., Infect Immun. (1998); 66: 2093-2098).

The effect of the carrier-protein dosage on the humoral response to the protein itself has also proven to be multifaceted. In human infants as it was reported that increasing the dosage of a tetravalent tetanus toxoid conjugate resulted in a decreased response to the tetanus carrier (Dagan et al., supra). Classical analysis of these effects of combination vaccines have been described as carrier induced epitopic suppression, which is not fully understood, but believed to result from an excess amount of carrier protein (Fattom, Vaccine 17: 126 (1999)). This appears to result in competition for Th-cells, by the B-cells to the carrier protein, and B-cells to the polysaccharide. If the B-cells to the carrier protein predominate, there are not enough Th-cells available to provide the necessary help for the B-cells specific to the polysaccharide. However, the observed immunological effects have been inconsistent, with the total amount of carrier protein in some instances increasing the immune response, and in other cases diminishing the immune response.

Hence there remain technical difficulties in combining multiple polysaccharide conjugates into a single, efficacious, vaccine formulation. It is thus an object of the present invention to develop an improved formulation of a multiple serotype Streptococcus pneumoniae polysaccharide conjugate vaccine.

### SUMMARY OF THE INVENTION

In one aspect the present invention is an improved Streptococcus pneumonia vaccine comprising 11 or more polysaccharides from different S. pneumonia serotypes conjugated to 2 or more carrier proteins, where the polysaccharides from serotypes 6B, 19F and 23F are conjugated to a first carrier protein and the remaining serotypes are conjugated to 1 or 2 secondary carrier proteins, and where the secondary carrier protein(s) are different from the first carrier protein. Preferably serotypes 6B and 23F are conjugated to the first carrier protein, and more preferably only serotype 6B is conjugated to the first carrier protein. In a preferred embodiment, one of the secondary carrier protein(s) is H. influenzae protein D. The present invention may further comprise S. pneumonia surface proteins, preferably from the PhtX family, the CbpX family, the CbpX truncate family and Ply.

In a related aspect, the present invention is an improved method to elicit a protective immune response to infants against S. pneumoniae by administering the polysaccharide conjugate vaccine of the present invention.

In another related aspect, the present invention is an improved method to elicit a protective immune response, that is, for the prevention or amelioration of pneumococcal infection in the elderly (e.g., pneumonia) and/or in infants (e.g., Otitis media), by administering the polysaccharide conjugated vaccine of the present invention and a S. pneumonia surface protein.

### BRIEF DESCRIPTION OF THE DRAWINGS

FIG. 1 is a graphical representation of the immune response to 12 different pneumococcal polysaccharides as determined by the geometric mean fold increase after polysaccharide immunization.

FIG. 2 shows the geometric mean IgG concentration [GMC] (μg/ml) and Opsonic Titres on day 14 (Post II) after immunization of adult rats with 1.0 μg PS-PD alone or combined in a tetravalent, pentavalent, heptavalent or decavalent vaccine.

FIG. 3 shows the GMC for 11 serotypes and PD (protein D) versus the dosage of 6B and 23F in one dimension, and the dosage of the 9 others in the second dimension. The trend is always the same for all serotypes and PD. Increasing the dosage of 6B and 23F has a dramatic effect on decreasing the immune response to tie remaining conjugates, even though the dosage of those conjugates is unchanged.

FIG. 4 shows a graph of the IgG GMC in infant rats versus the total amount of PD immunized for 11 serotypes. (i.e., by summing all the PD from each component at each dose). The general trend is that as the dosage of carrier protein increases, there is a decrease in the IgG response to all polysaccharides, and to PD itself. This overall trend is strong evidence of carrier-induced epitopic suppression. However, the fact that the curve is not monotonous is an indication that there is another factor involved which appears to depend on Serotype 6B.

### DETAILED DESCRIPTION OF THE INVENTION

The present invention provides an optimal formulation of multiple-serotype Streptococcus pneumoniae polysaccharide conjugate vaccines, by judicious selection of various polysaccharides conjugated to different, or alternate, carrier proteins. The invention is based on the fact that polysaccharide conjugates of one serotype may influence, or modulate, the immune response observed for other (serotype) polysaccharide conjugates. Thus, an optimal multi-valent polysaccharide conjugate vaccine can be prepared by putting different S. pneumonia polysaccharides, with different immune regulatory properties, on alternative carrier proteins.

The present invention is based on the combination of several factors: (i) the dosage-response curve to polysaccharides is frequently bell-shaped (Gaussian), with the maximal response at a dosage distinctive for each polysaccharide (i.e., serotype or structure); (ii) the immunogenicity of certain polysaccharides is regulated with age in humans and in animal models; (iii) the combination of S. pneumoniae polysaccharide conjugates into multivalent formulations often results in a decrease in immunogenicity of one or more components of the vaccine; (iv) however, certain polysaccharide conjugates result in an enhanced immune response when combined; (v) polysaccharides from serotypes 6B and 23F, and to a lesser degree 19F can regulate the immune response of other polysaccharides (i.e., other serotypes) when they are conjugated to a common carrier protein.

Thus the present invention is based on the complex relationship of all the above and, in contrast to prior studies, concludes that the bell-shaped dosage-response curve (i.e., which denotes peak immunogenicity) of polysaccharide-protein conjugates is heavily influenced by the quantity and nature of other polysaccharides. This immunological effect is referred to as modulation. Moreover, it has been discovered that the modulation of polysaccharide conjugates occurs through a common carrier protein. That is, a few polysaccharide conjugates may modulate the immune response to different polysaccharide conjugates, so long as they have a common carrier protein. Thus as noted above, the invention is based on the judicious selection polysaccharides, to determine which polysaccharides should be conjugated to the same or different carrier proteins.

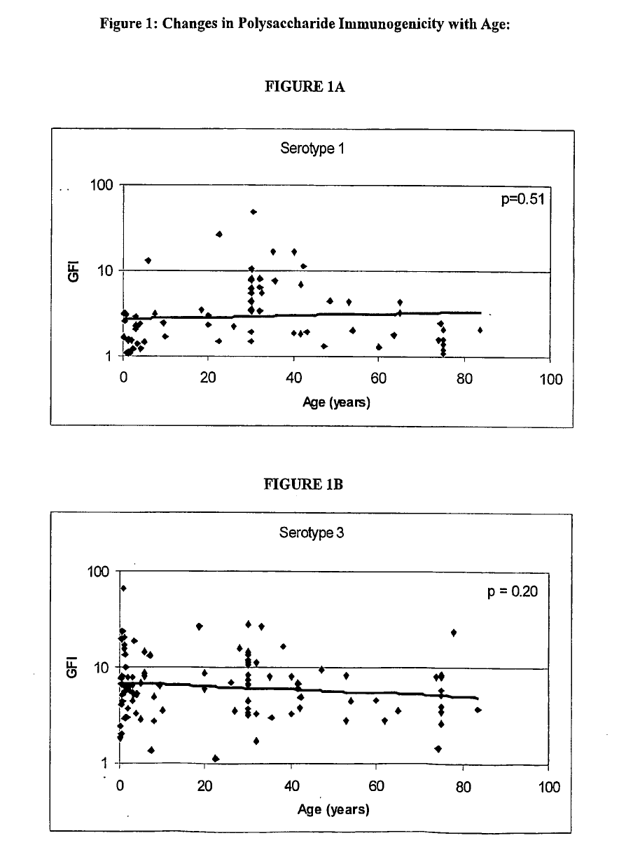

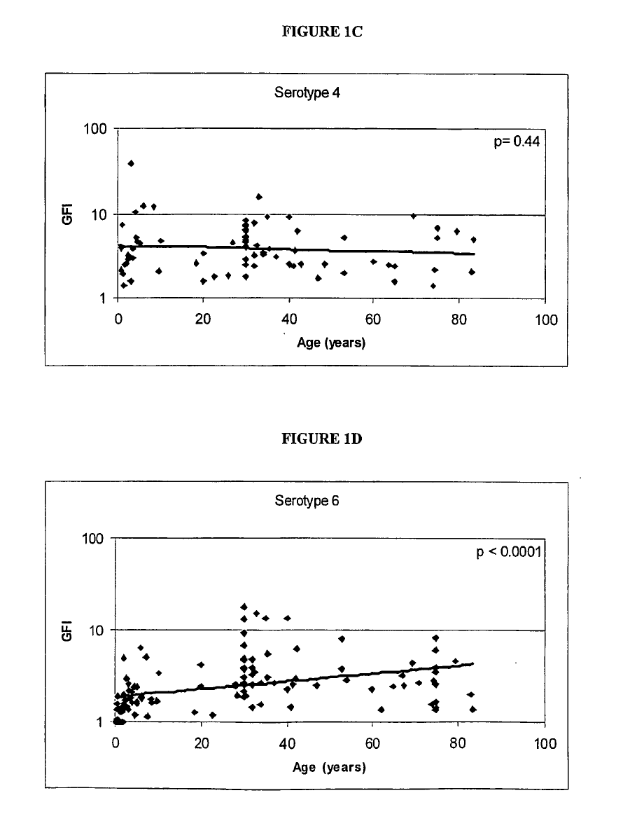

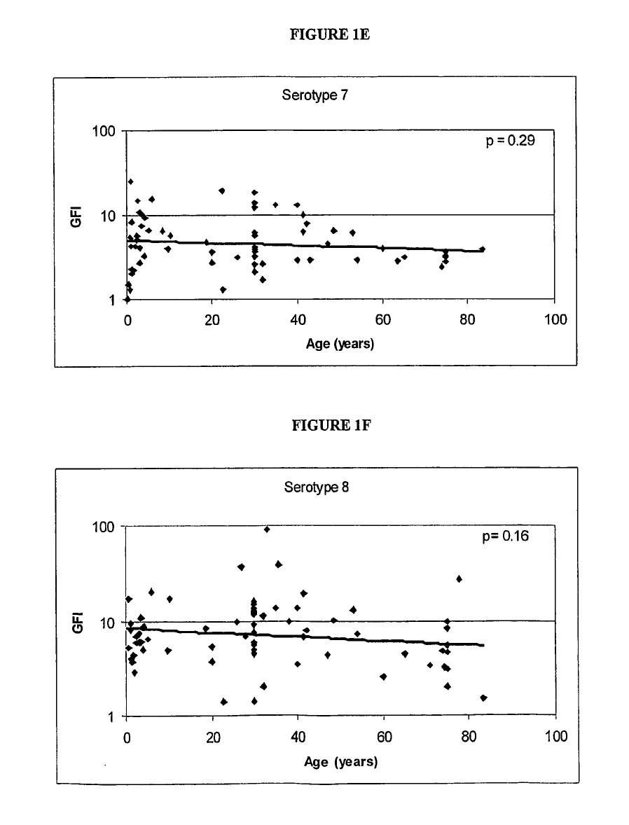

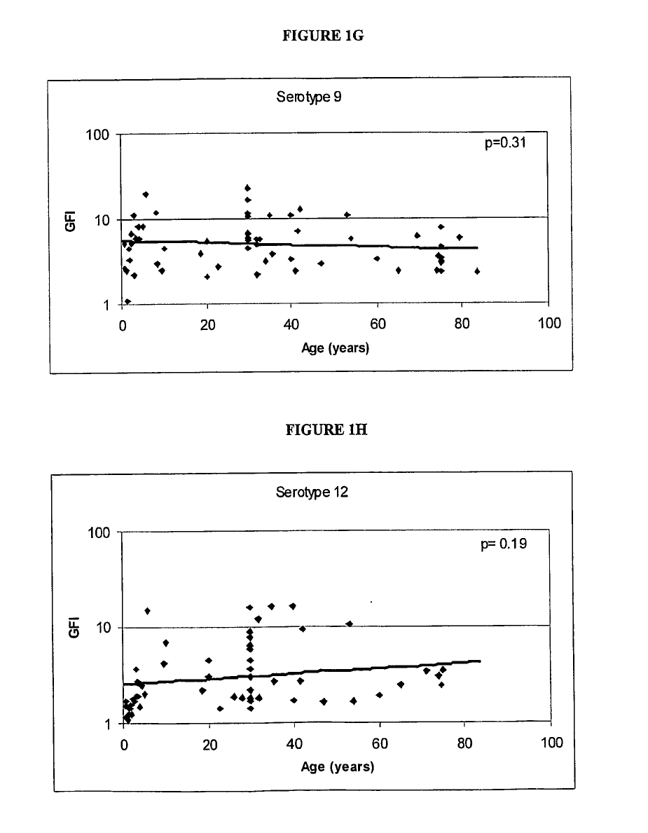

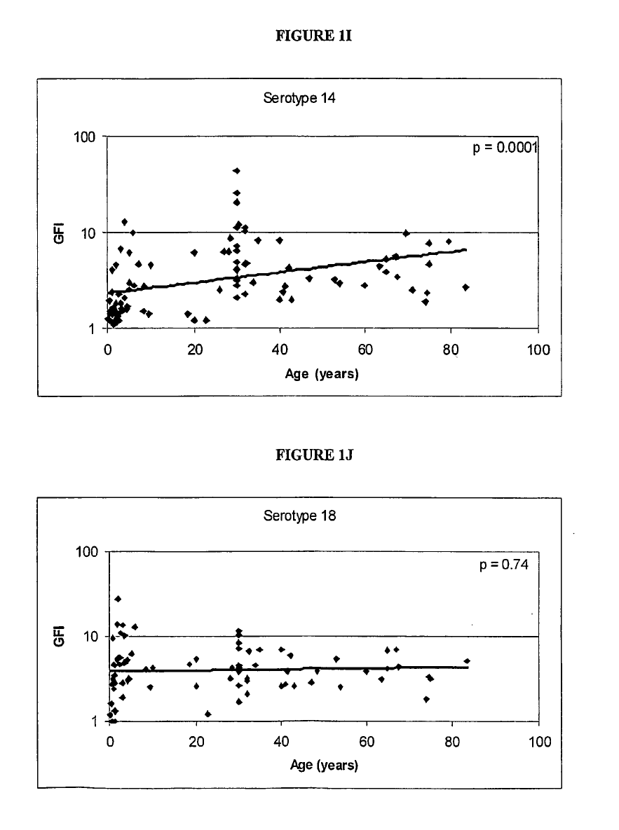

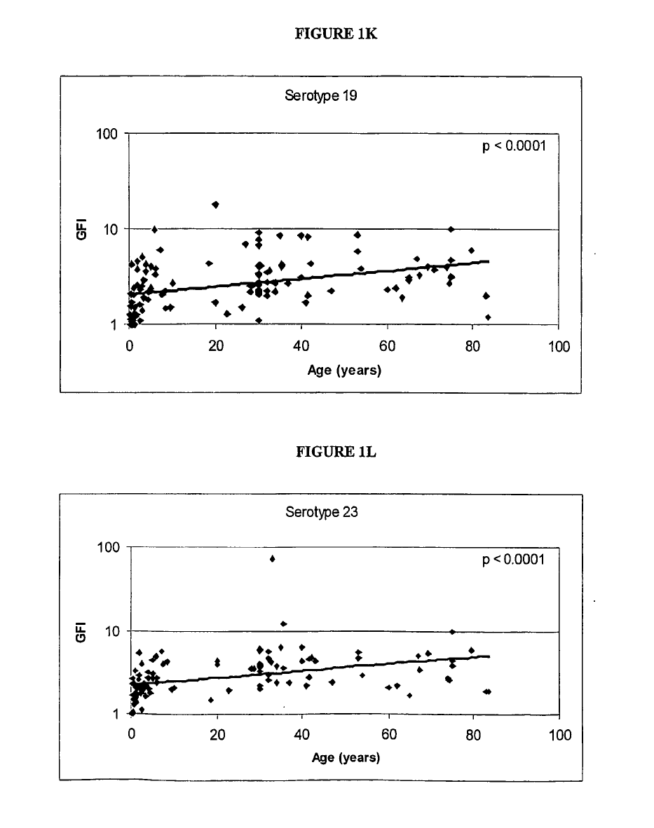

As shown in more detail below: (a) certain S. pneumonia polysaccharides (PS), when conjugated, are strongly regulated with age, in particular serotypes 6B, 14, 19F and 23F. Serotypes 8, 12 and 18C are weakly regulated with age. Serotypes 1, 2, 3, 4, 5, 7F and 9V are not regulated with age (see FIG. 1).

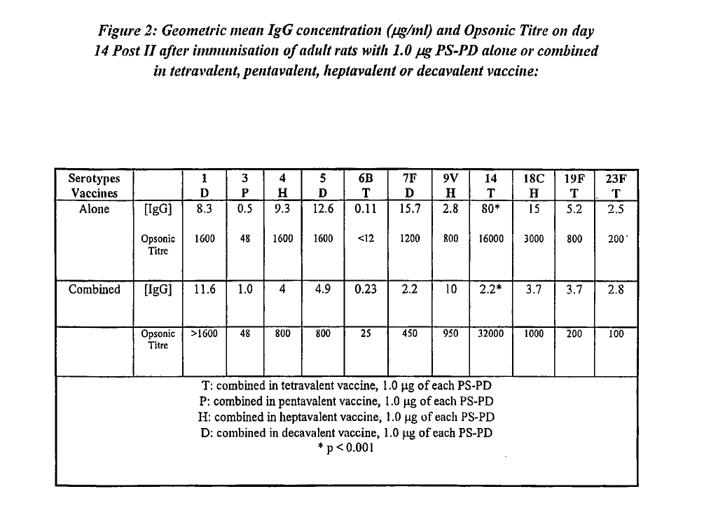

In addition (b), polysaccharides 1, 3, 6B, 9V and 23F, when combined into an 11-valent multiformulation, showed an increase in the immune response elicited, as compared to a monovalent polysaccharide conjugate. In contrast, serotype 14 showed a significant decrease in the multivalent formulation (see FIG. 2).

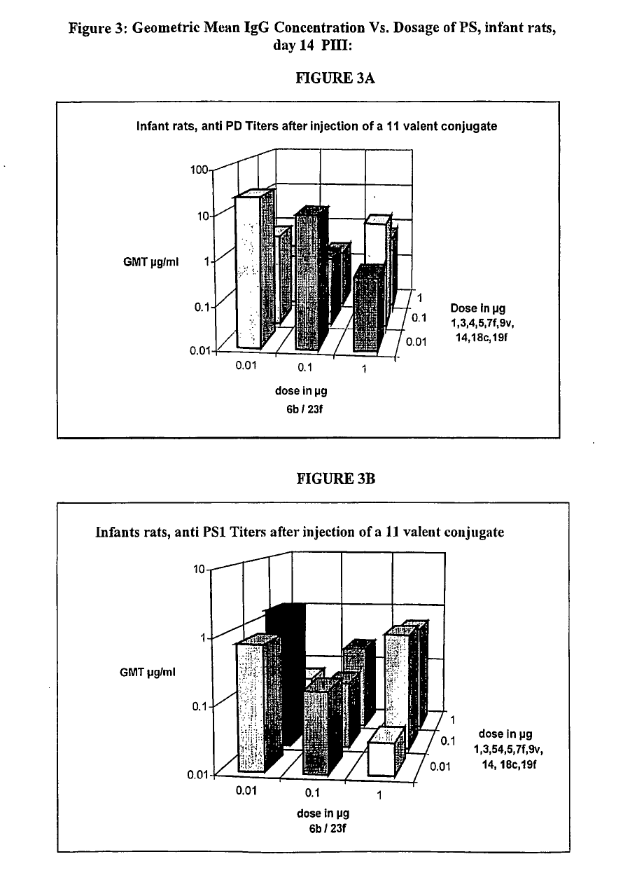

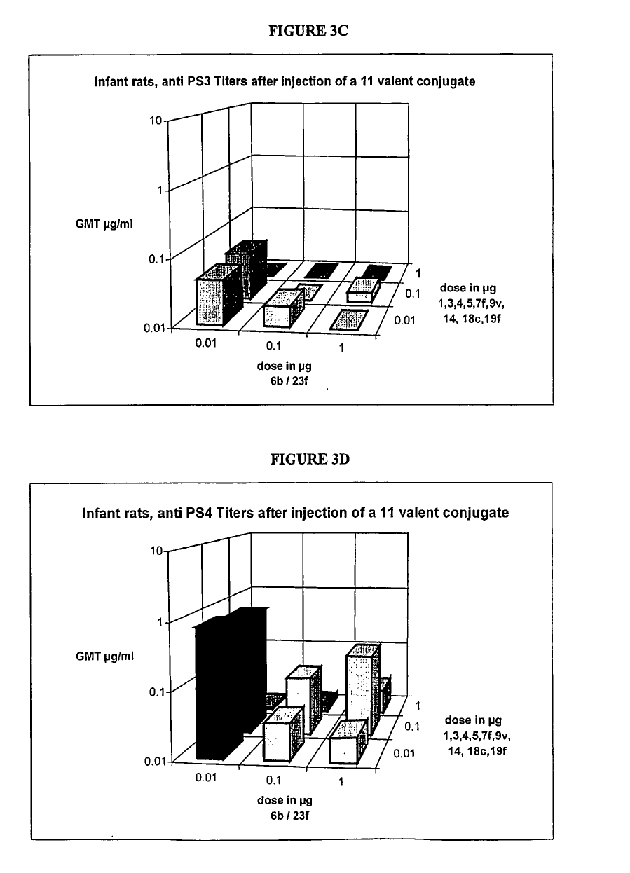

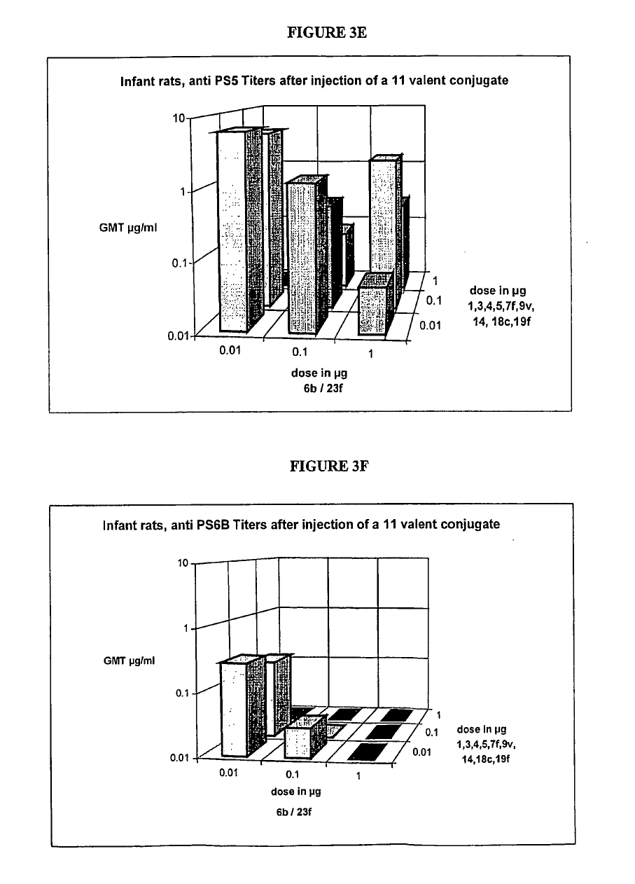

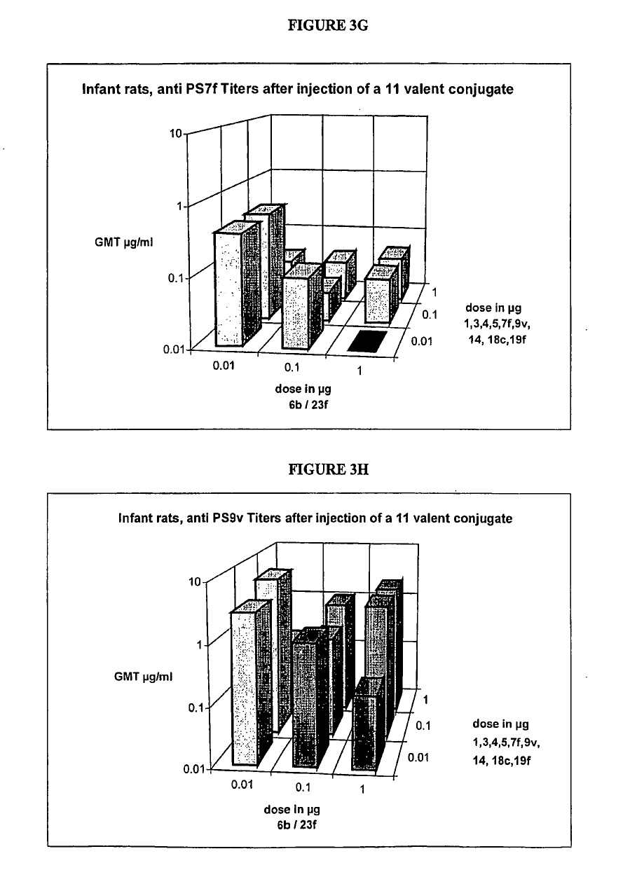

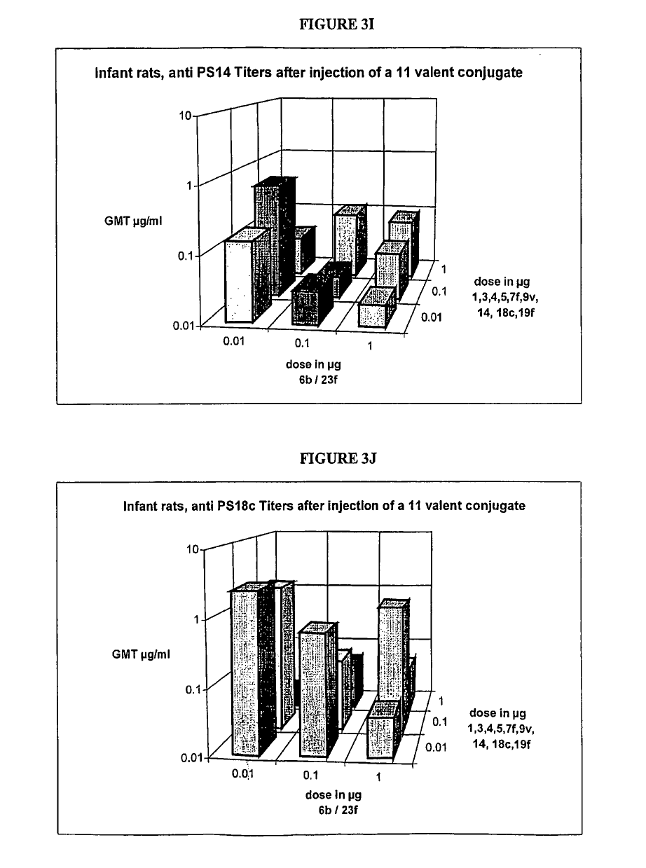

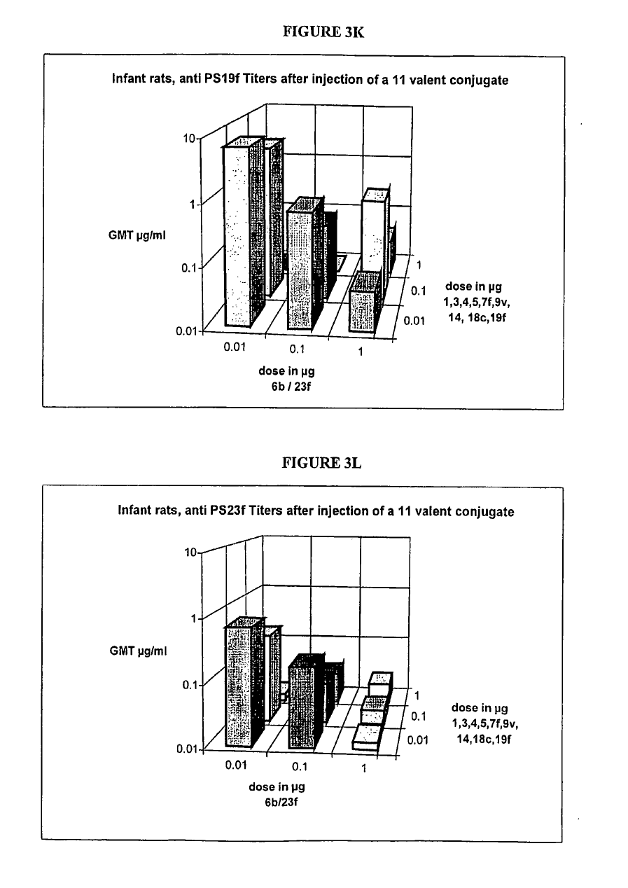

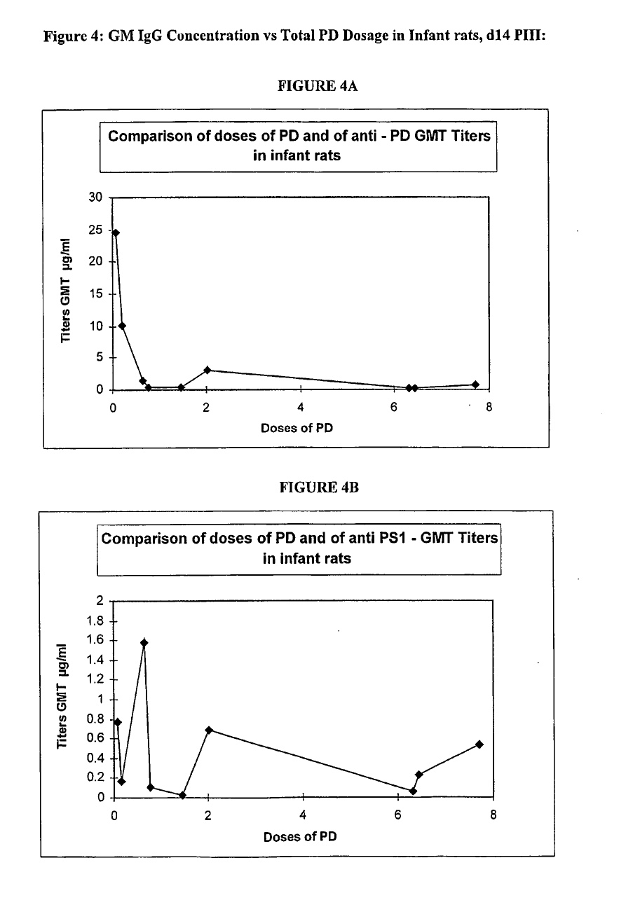

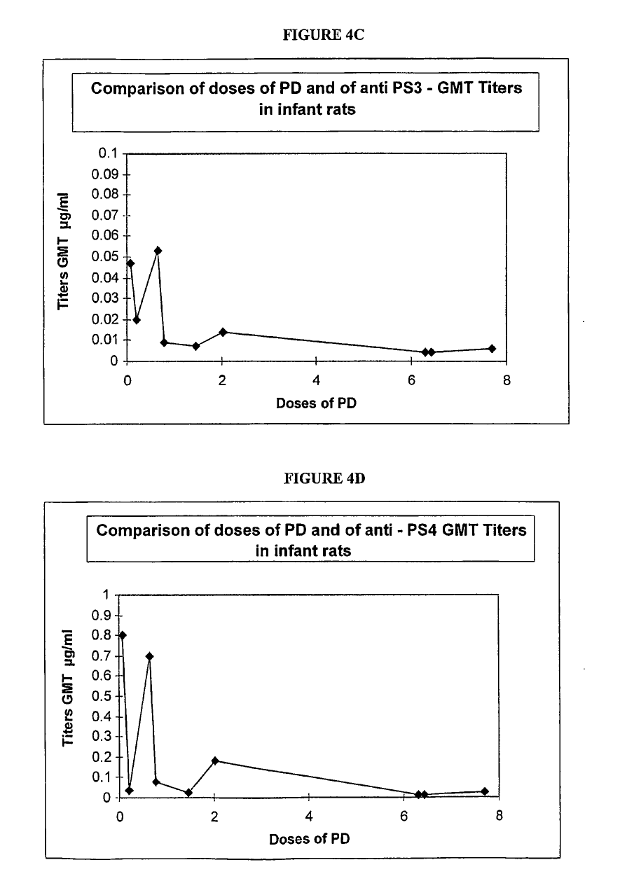

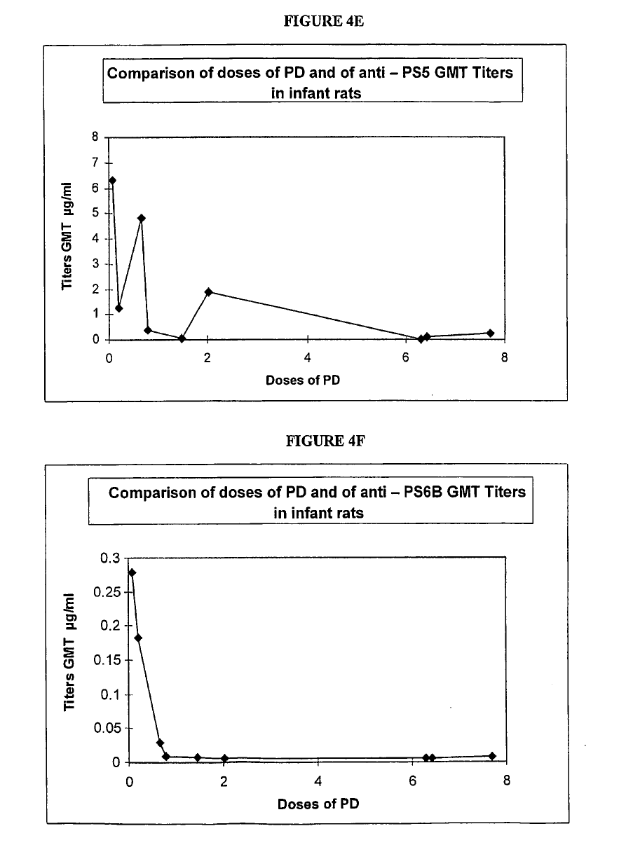

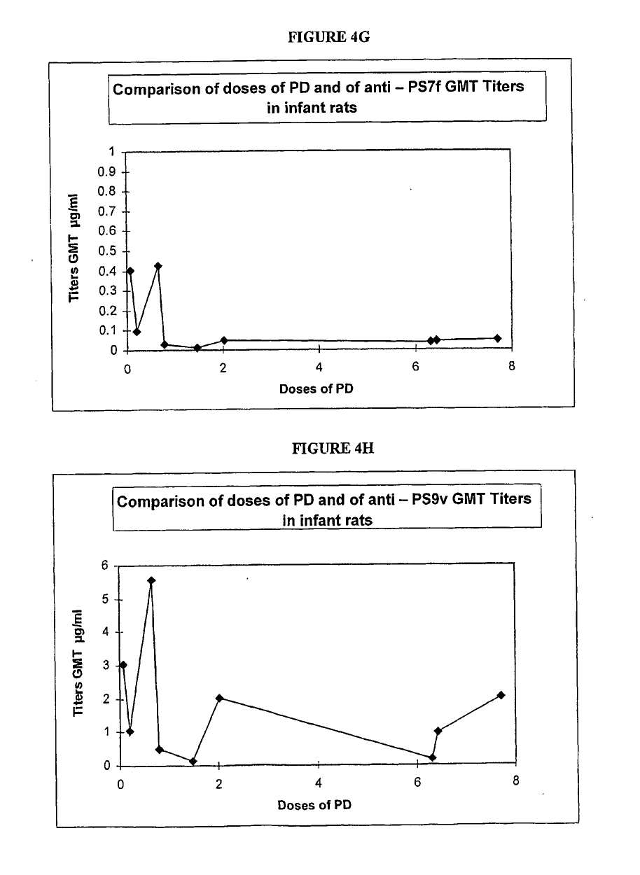

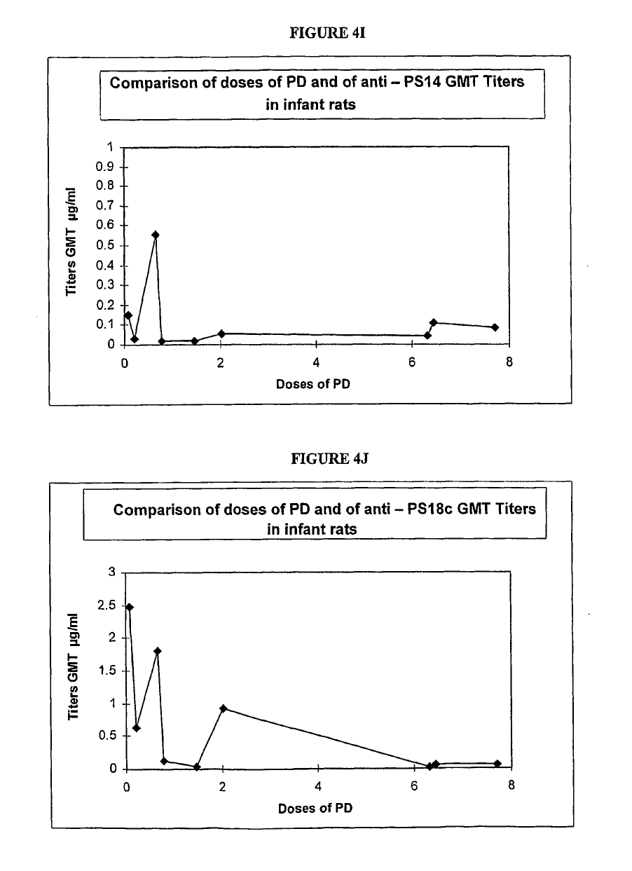

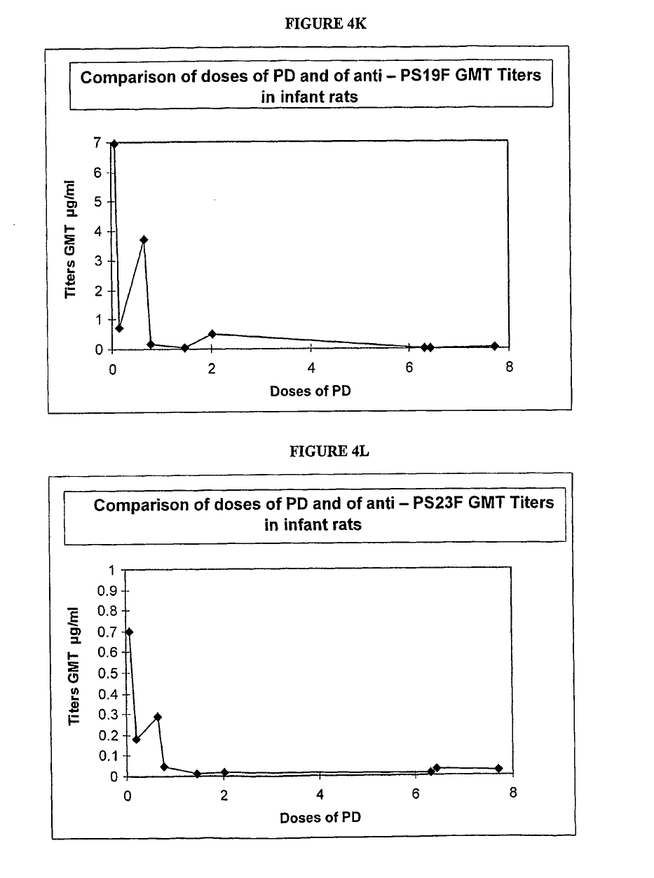

Moreover (c), polysaccharides from serotypes 6B and 23F, and to a lesser degree 19F can regulate the immune response of other polysaccharides (i.e., other serotypes) if they are conjugated to a common carrier protein (see FIGS. 3 and 4).

Thus in one embodiment, the present invention comprises polysaccharides 6B, 19F and 23F conjugated with one (a first) carrier protein, and the remaining polysaccharides are conjugated to an alternative (or secondary) carrier protein(s), with the proviso that the primary and secondary carrier proteins are different. Preferably, polysaccharides 6B and 23F are conjugated with the same carrier protein, and the remaining polysaccharides are conjugated to a secondary carrier protein(s). More preferably, only polysaccharide 6B is conjugated to a primary (first) carrier protein and the remaining polysaccharides are conjugated to a secondary carrier protein(s).

The primary carrier protein need not be limited to a specific embodiment, but may include proteins or fragments thereof of DT (Diphtheria toxoid), TT (Tetanus toxoid), DT crm197 (a DT mutant), other DT point mutants, (e.g.at position Glu-148, see, e.g., U.S. Pat. No. 4,709,017, WO93/25210, WO95/33481), FragC (fragment of TT), Ply (pneumolysin and mutants thereof), PhtA, PhtB, PhtD, PhtE, (Pht A-E are described in more detail below) OnipC (from N. meningitidis), PorB (from N. meningitidis), etc. Preferably it is DT, TT or crm 197. More preferably it is DT.

The secondary carrier protein(s) will also be selected from the group consisting of PD (Haemophilus influenzae protein D—see, e.g., EP 0 594 610 B), DT, TT, DT crm 197, FragC, Ply, PhtA, PhtB, PhtD, PhtE, OmpC, PorB, etc. It is contemplated that 2 different secondary carrier proteins may be used, but preferably, only one secondary carrier protein is to be used in the present invention.

The number of S. pneumoniae polysaccharides can range from 11 different serotypes (or “V”, valences) to 23 different serotypes (23V). Preferably it is 11, 13 or 16 different serotypes. In another embodiment of the invention, the vaccine may comprise conjugated S. pneumoniae polysaccharides and unconjugated S. pneumoniae polysaccharides. Preferably, the total number of polysaccharide serotypes is less than or equal to 23. For example, the invention may comprise 11 conjugated serotypes and 12 unconjugated polysaccharides. In a similar manner, the vaccine may comprise 13 or 16 conjugated polysaccharides and 10, or 7 respectively, unconjugated polysaccharides.

Preferably the multivalent pneumococcal vaccine of the invention will be selected from the following serotypes 1, 2, 3, 4, 5, 6B, 7F, 8, 9N, 9V, 10A, 11A, 12F, 14, 15B, 17F, 18C, 19A, 19F, 20, 22F, 23F and 33F, although it is appreciated that one or two other serotypes could be substituted depending on the age of the recipient receiving the vaccine and the geographical location where the vaccine will be administered. For example, an 11-valent vaccine may comprise polysaccharides from serotypes 1, 3, 4, 5, 6B, 7F, 9V, 14, 18C, 19F and 23F. A 13-valent pediatric (infant) vaccine may also include serotypes 6A and 19A, whereas a 13-valent elderly vaccine may include serotypes 8 and 12F.

Preferably, the Streptococcus polysaccharides of the invention are depolymerized (sized) to a final range of 100-500 kD. Thus, another feature of the present invention is the ratio of carrier protein to polysaccharide. For the conjugated polysaccharides, the ratio of carrier protein to polysaccharide (P/PS) will be greater than 0.5 (i.e., >0.5, and up to 1.7) (w/w) for at least seven serotypes. Preferably the ratio is □0.70 to 1.5 (e.g., for at least serotypes 6B, 19F, 23F). More preferably the range will be 0.8 to 1.5 (e.g., for at least serotypes 6B, 19F, 23F). Most preferably still, the ratio of P/PS will at least approach 1 (e.g., 0.9-1.1) for one or more serotypes of the invention (e.g., 4).

A related feature of the present invention is that the level of unconjugated (free) carrier protein is less than 10% of the total amount of carrier protein, and that the level of unconjugated polysaccharide is less than 10% of the total amount of polysaccharide, for each serotype.

The polysaccharides may be linked to the carrier protein(s) by any known method (for example, by Likhite, U.S. Pat. No. 4,372,945 by Armor et al., U.S. Pat. No. 4,474,757, and Jennings et al., U.S. Pat. No. 4,356,170). Preferably, CDAP conjugation chemistry is carried out (see WO95/08348).

In CDAP, the cyanylating reagent 1cyano-dimethylaminopyridinium tetrafluoroborate (CDAP) is preferably used for the synthesis of polysaccharide-protein conjugates. The cyanilation reaction can be performed under relatively mild conditions, which avoids hydrolysis of the alkaline sensitive polysaccharides. This synthesis allows direct coupling to a carrier protein.

The polysaccharide is solubilized in water or a saline solution. CDAP is dissolved in acetonitrile and added immediately to the polysaccharide solution. The CDAP reacts with the hydroxyl groups of the polysaccharide to form a cyanate ester. After the activation step, the carrier protein is added. Amino groups of lysine react with the activated polysaccharide to form an isourea covalent link. After the coupling reaction, a large excess of glycine is then added to quench residual activated functional groups. The product is then passed through a gel permeation column to remove unreacted carrier protein and residual reagents.

In another embodiment, the S. pneumonia conjugates may be combined with other polysaccharides, for example, N. meningitidis types A, C, W, Y, H. influenzae type B, S. aureus, S. epidermidis, Group B Streptococcus, Group A Streptococcus, etc. Preferably it is N. meningitidis (types A and/or C are most preferred) and/or H. influenzae type B. Alternatively, the S. pneumonia conjugates of the invention may be combined with viral antigens, e.g., inactivated polio virus (IPV), influenza (inactivated, split subunit (e.g., F, G antigens)), etc. In another alternative, the S. pneumonia conjugates may be administered concomitantly with DTPa (diphtheria, tetanus, acellular pertussis) vaccines and DTPa combination vaccines (DTPa±/Hepatitis B±IPV±H. influenzae type B). Preferred DTPa vaccines have 25 Lf or less of Diphtheria toxoid. These additional antigens may be in liquid form or lyophilized form.

In yet another embodiment, die present invention is an improved method to elicit a (protective) immune response in infants (0-2 years old) by administering a safe and effective amount of the vaccine of the invention. Further embodiments of the present invention include the provision of the antigenic S. pneumoniae conjugate compositions of the invention for use in medicine and the use of the S. pneumoniae conjugates of the invention in the manufacture of a medicament for the prevention (or treatment) of pneumococcal disease.

The present invention further provides an improved vaccine for the prevention or amelioration of pneumococcal infection in infants (e.g., Otitis media), by relying on the addition of pneumococcal proteins to S. pneumoniae conjugate compositions of the invention. Preferably the pneumococcal protein is from the PhtX family (see below) to which may be added further proteins. Such additional pneumococcal proteins may comprise CbpX, CbpX truncates and Ply (see below), with the proviso that the selected S. pneumoniae surface proteins are different from the first and secondary carrier proteins. One or more Moraxella catarrhalis protein antigens can also be included in the combination vaccine. Thus, the present invention is an improved method to elicit a (protective) immune response against Otitis media in infants.

In yet another embodiment, the present invention is an improved method to elicit a (protective) immune response in the elderly population (in the context of the present invention a patient is considered elderly if they are 50 years or over in age, typically over 55 years and more generally over 60 years) by administering a safe and effective amount of the vaccine of the invention, preferably in conjunction with one, two, or possibly three S. pneumoniae surface proteins, with the proviso that the selected S. pneumoniae surface proteins are different from the first and secondary carrier proteins. Preferably the pneumococcal protein is from the PhtX family (see below) to which may be added Ply and optionally CbpX or CbpX truncates (see below).

The Streptococcus pneumoniae proteins of the invention are either surface exposed, at least during part of the life cycle of the pneumococcus, or are proteins which are secreted or released by the pneumococcus. Preferably the proteins of the invention are selected from the following categories, such as proteins having a Type II Signal sequence motif of LXXC (where X is any amino acid, e.g., the polyhistidine triad family (PhtX)), choline binding proteins (CbpX), proteins having a Type I Signal sequence motif (e.g., Sp101), proteins having a LPXTG motif (where X is any amino acid, e.g., Sp128, Sp130), and toxins (e.g., Ply). Preferred examples within these categories (or motifs) are the following proteins, or immunologically functional equivalents thereof.

Preferably, the immunogenic composition of the invention comprises one or more proteins selected from the group consisting of the Poly Histidine Triad family (PhtX), Choline Binding Protein family (CbpX), CbpX truncates, LytX family, LytX truncates, CbpX truncate-LytX truncate chimeric proteins (or fusions), pneumolysin (Ply), PspA, PsaA, Sp128, Sp101, Sp130, Sp]25 and Sp133. However, if CbpX is PspC, then the second protein is not PspA or PsaA. More preferably, the immunogenic composition comprises 2 or more proteins selected from the group consisting of the Poly Histidine Triad family (PhtX), Choline Binding Protein family (CbpX), CbpX truncates, LytX family, LytX truncates, CbpX truncate-LyCX truncate chimeric proteins (or fusions), pneumolysin (Ply), PspA, PsaA, and Sp128. More preferably still, the immunogenic composition comprises 2 or more proteins selected from the group consisting of the Poly Histidine Triad family (PhtX), Choline Binding Protein family (CbpX), CbpX truncates, and pneumolysin (Ply).

The Pht (Poly Histidine Triad) family comprises proteins PhtA, PhtB, PhtD, and PhtE. The family is characterized by a lipidation sequence, two domains separated by a proline-rich region and several histidine triads, possibly involved in metal or nucleoside binding or enzymatic activity, (3-5) coiled-coil regions, a conserved N-terminus and a heterogeneous C terminus. It is present in all strains of pneumococci tested. Homologous proteins have also been found in other Streptococci and Neisseria. Preferred members of the family comprise PhtA, PhtB and PhtD. More preferably, it comprises PhtA or PhtD. Most preferably it comprises PhtD. It is understood, however, that the terms Pht A, B, D, and E refer to proteins having sequences disclosed in the citations below as well as naturally-occurring (and man-made) variants thereof that have a sequence homology that is at least 90% identical to the referenced proteins. Preferably it is at least 95% identical and most preferably it is 97% identical.

With regards to the PhtX proteins, PhtA is disclosed in WO 98/18930, and is also referred to Sp36. As noted above, it is a protein from the polyhistidine triad family and has the type II signal motif of LXXC. PhtD is disclosed in WO 00/37105, and is also referred to Sp036D. As noted above, it also is a protein from the polyhistidine triad family and has the type II LXXC signal motif. PhtB is disclosed in WO 00/37105, and is also referred to Sp036B. Another member of the PhtB family is the C3-Degrading Polypeptide, as disclosed in WO 00/17370. This protein also is from the polyhistidine triad family and has the type II LXXC signal motif. A preferred immunologically functional equivalent is the protein Sp42 disclosed in WO 98/18930. A PhtB truncate (approximately 79 kD) is disclosed in WO99/15675 which is also considered a member of the PhtX family. PhtE is disclosed in WO 00/30299 and is referred to as BVH-3.

Concerning the Choline Binding Protein family (CbpX), members of that family were originally identified as pneumococcal proteins that could be purified by choline-affinity chromatography. All of the choline-binding proteins are non-covalently bound to phosphoryleholine moieties of cell wall teichoic acid and membrane-associated lipoteichoic acid. Structurally, they have several regions in common over the entire family, although the exact nature of the proteins (amino acid sequence, length, etc.) can vary. In general, choline binding proteins comprise an N terminal region (N), conserved repeat regions (R1 and/or R2), a proline rich region (P) and a conserved choline binding region (C), made up of multiple repeats, that comprises approximately one half of the protein. As used in this application, the term “Choline Binding Protein family (CbpX)” is selected from the group consisting of Choline Binding Proteins as identified in WO97/41151, PbcA, SpsA, PspC, CbpA, CbpD, and CbpG. CbpA is disclosed in WO97/41151. CbpD and CbpG are disclosed in WO0/29434. PspC is disclosed in WO97/09994. PbcA is disclosed in WO98/21337.SpsA is a Choline binding protein disclosed in WO 98/39450. Preferably the Choline Binding Proteins are selected from the group consisting of CbpA, PbcA, SpsA and PspC.

Another preferred embodiment is CbpX truncates wherein “CbpX” is defined above and “truncates” refers to CbpX proteins lacking 50% or more of the Choline binding region (C). Preferably such proteins lack the entire choline binding region. More preferably, such protein truncates lack (i) the choline binding region and (ii) retain the proline rich region and at least one repeat region (R1 or R2). More preferably still, the truncate has 2 repeat regions (R1 and R2). Examples of such preferred embodiments are NR1×R2, NR1×R2P, R1×R2P and R1×R2 as illustrated in WO99/51266 or WO99/51188, however, other choline binding proteins lacking a similar choline binding region are also contemplated within the scope of this invention.

The LytX family is membrane associated proteins associated with cell lysis. The N-terminal domain comprises choline binding domain(s), however the LytX family does not have all the features found in the CbpA family noted above and thus for the present invention, the LytX family is considered distinct from the CbpX family. In contrast with the CbpX family, the C-terminal domain contains the catalytic domain of the LytX protein family. The family comprises LytA, B and C. With regards to the LytX family, LytA is disclosed in Ronda et al., Eur J Biochem, 164:621-624 (1987). LytB is disclosed in WO 98/18930, and is also referred to as Sp46. LytC is also disclosed in WO 98/18930, and is also referred to as Sp91. A preferred member of that family is LytC.

Another preferred embodiment are LytX truncates wherein “LytX” is defined above and “truncates” refers to LytX proteins lacking 50% or more of the Choline binding region. Preferably such proteins lack the entire choline binding region. Yet another preferred embodiment of this invention are CbpX truncate-LytX truncate chimeric proteins (or fusions). Preferably this comprises NR1xR2 (or R1xR2) of CbpX and the C-terminal portion (Cterm, i.e., lacking the choline binding domains) of LytX (e.g., LytCCterm or Sp91Ctern). More preferably CbpX is selected from the group consisting of CbpA, PbcA, SpsA and PspC. More preferably still, it is CbpA. Preferably, LytX is LytC (also referred to as Sp91). Another embodiment of the present invention is a PspA or PsaA truncates lacking the choline binding domain (C) and expressed as a fusion protein with LytX. Preferably, LytX is LytC.

Pneumolysin is a multifunctional toxin with a distinct cytolytic (hemolytic) and complement activation activities (Rubins et al., Am . Respi. Cit Care Med, 153:1339-1346 (1996)). The toxin is not secreted by pneumococci, but it is released upon lysis of pneumococci under the influence of autolysin. Its effects include e.g., the stimulation of the production of inflammatory cytokines by human montocytes, the inhibition of the beating of cilia on human respiratory epithelial, and the decrease of bactericidal activity and migration of neutrophils. The most obvious effect of pneumolysin is in the lysis of red blood cells, which involves binding to cholesterol. Because it is a toxin, it needs to be detoxified (i.e., non-toxic to a human when provided at a dosage suitable for protection) before it can be administered in vivo. Expression and cloning of wild-type or native pneumolysin is known in the art. See, for example, Walker et al. (Infect Immun, 55:1184-1189 (1987)), Mitchell et al. (Biochim Biophys Acta, 1007:67-72 (1989) and Mitchell et al (WAR, 18:4010 (1990)). Detoxification of ply can be conducted by chemical means, e.g., subject toGMBS, or formalin or glutarahdehye treatment or a combination of both. Such methods are well known in the art for various toxins. Alternatively, ply can be genetically detoxified. Thus, the invention encompasses derivatives of pneumococcal proteins which may be, for example, mutated proteins. The term “mutated” is used herein to mean a molecule which has undergone deletion, addition or substitution of one or more amino acids using well known techniques for site directed mutagenesis or any other conventional method. For example, as described above, a mutant ply protein may be altered so that it is biologically inactive whilst still maintaining its immunogenic epitopes, see, for example, WO90/06951, Berryetal. (Infect Immun, 67:981-985(1999)) and WO99/03884. As used herein, it is understood that the term “Ply” refers to mutated or detoxified pneumolysin suitable for medical use (i.e., non toxic).

With regards to PsaA and PspA, both are know in the art. For example, PsaA and transmembrane deletion variants thereof have been described by Berry & Paton, Infect Immun December 1996;64(12):5255-62. PspA and transmembrane deletion variants thereof have been disclosed in, for example, U.S. Pat. No. 5,804,193, WO 92/14488, and WO 99/53940.

Sp128 and Sp130 are disclosed in WO00/76540. Sp125 is an example of a pneumococcal surface protein with the Cell Wall Anchored motif of LPXTG (where X is any amino acid). Any protein within this class of pneumococcal surface protein with this motif has been found to be useful within the context of this invention, and is therefore considered a further protein of the invention. Sp125 itself is disclosed in WO 98/18930, and is also known as ZmpB—a zinc metalloproteinase. Sp101 is disclosed in WO 98/06734 (where it has the reference # y85993). It is characterized by a Type I signal sequence. Sp133 is disclosed in WO 98/06734 (where it has the reference # y85992). It is also characterized by a Type I signal sequence.

Examples of preferred Moraxella catarrhalis protein antigens which can be included in a combination vaccine (especially for the prevention of otitis media) are: OMP 106 [WO 97/41731 (Antex) & WO 96/34960 (PMC)]; OMP21; LbpA &/or LbpB [WO 98/55606 (PMC)]; TbpA &/or TbpB [WO 97/13785 & WO 97/32980 (PMC)]; CopB [Helminen M E, et al. (1993) Infect. Immun. 61:2003-201 0]; UspA1 and/or UspA2 [WO 93/03 761 (University of Texas)]; OmpCD; HasR (PCT/EP99/03824); PilQ (PCT/EP99/03823); OMP85 (PCT/EP00/01468); lipo06 (GB 9917977.2); lipo10 (GB 9918208.1); lipo11 (GB 9918302.2); lipo18 (GB 9918038.2); P6 (PCT/EP99/03038); D15 (PCT/EP99/03822); OmplA1 (PCT/EP99/0678 1); Hly3 (PCT/EP99/03257); and OmpE. Examples of non-typeable Haemophilus influenzae antigens which can be included in a combination vaccine (especially for the prevention of otitis media) include: Fimbrin protein [(U.S. Pat. No. 5,766,608—Ohio State Research Foundation)] and fusions comprising peptides therefrom [eg LB1(f) peptide fusions; U.S. Pat. No. 5,843,464 (OSU) or WO 99/64067]; OMP26 [WO 97/01638 (Cortecs)]; P6 [EP 281673 (State University of New York)]; TbpA and/or TbpB; Hia; Hsf; Hin47; Hif; Hmw1; Hmw2; Hmw3; Hmw4; Hap; D15 (WO 94/12641); P2; and PS (WO 94/26304).

As noted above, the proteins of the invention may also be beneficially combined. Preferred combinations include, but are not limited to, PhtD+NR1×R2, PhtD+NR1×R2P, PhtD+NR1×R2-Sp91Cterm chimeric or fusion proteins, PhtD+Ply, PhtD+Sp128, PhtD+PsaA, PhtD+PspA, PhtA+NR1×R2, PhtA+NR1×R2P, PhtA+NR1×R2-Sp91Cterm chimeric or fusion proteins, PhtA+Ply, PhtA+Sp128, PhtA+PsaA, PhtA+PspA, NR1×R2+LytC, NR1×R2P+PspA, NR1×R2+PspA, NR1×R2P+PsaA, NR1×R2+PsaA, NR¹×R2+Sp128, R1×R2+LytC, R¹×R2+PspA, R1×R2+PsaA, R1×R2+Sp128, R1×R2+PhtD, R1×R2+PhtA. Preferably, NR1×R2±P (or R1×R2±P) is from CbpA or PspC. More preferably it is from CbpA. Other combinations include 3 protein combinations such as PhtD+NR1×R2P +Ply, PhtD+NR1×R2+Ply, PhtA+NR1×R2+Ply and PhtA+NR1×R2P+Ply.

The vaccines of the present invention are preferably adjuvanted. Suitable adjuvants include an aluminum salt such as aluminum hydroxide gel (alum) or aluminum phosphate, but may also be a salt of calcium, magnesium, iron or zinc, or may be an insoluble suspension of acylated tyrosine, or acylated sugars, cationically or anionically derivatized polysaccharides, or polyphosphazenes. When adjuvanted with aluminum salts, the ratio of aluminum salt to polysaccharide is less than 10:1 (w/w). Preferably it is less than 8:1 and more than 2:1.

It is preferred that the adjuvant be selected to be a preferential inducer of a TH1 type of response. Such high levels of Th1-type cytokines tend to favor the induction of cell mediated immune responses to a given antigen, whilst high levels of Th2-type cytokines tend to favor the induction of humoral immune responses to the antigen.

It is important to remember that the distinction of Th1 and Th2-type immune response is not absolute. In reality an individual will support an immune response which is described as being predominantly Th1 or predominantly Th2. However, it is often convenient to consider the families of cytokines in terms of that described in murine CD4+ve T cell clones by Mosmann and Coffman (Mosmann, T. R. and Coffman, R L. (1989) TH1 and TH2 cells: different patterns of lymphokine secretion lead to different functional properties. Annual Review of Immunology, 7, p1 45-173). Traditionally, Th1-type responses are associated with the production of the INF-γ and IL-2 cytokines by T-lymphocytes. Other cytokines often directly associated with the induction of Th1-type immune responses are not produced by T-cells, such as IL-12. In contrast, Th2-type responses are associated with the secretion of IL-4, IL-5, IL-6, IL-10. Suitable adjuvant systems which promote a predominantly Th1 response include: Monophosphoryl lipid A or a derivative thereof, particularly 3-de-O-acylated monophosphoryl lipid A (3D-MPL) (for its preparation see GB 2220211 A); and a combination of monophosphoryl lipid A, preferably 3-de-O-acylated monophosphoryl lipid A, together with either an aluminum salt (for instance aluminum phosphate or aluminum hydroxide) or an oil-in-water emulsion. In such combinations, antigen and 3D-MPL are contained in the same particulate structures, allowing for more efficient delivery of antigenic and immunostimulatory signals. Studies have shown that 3D-MPL is able to further enhance the immunogenicity of an alum-adsorbed antigen [Thoelen et al. Vaccine (1998) 16:708-14; EP 689454-B1].

An enhanced system involves the combination of a monophosphoryl lipid A and a saponin derivative, particularly the combination of QS21 and 3D-MPL as disclosed in WO 94/00153, or a less reactogenic composition where the QS21 is quenched with cholesterol as disclosed in WO 96/33739. A particularly potent adjuvant formulation involving QS21, 3D-MPL and tocopherol in an oil in water emulsion is described in WO 95/17210, and is a preferred formulation. Preferably the vaccine additionally comprises a saponin, more preferably QS21. The formulation may also comprise an oil in water emulsion and tocopherol (WO 95/17210). The present invention also provides a method for producing a vaccine formulation comprising mixing a protein of the present invention together with a pharmaceutically acceptable excipient, such as 3D-MPL. Unmethylated CpG containing oligonucleotides (WO 96/02555) are also preferential inducers of a TH1 response and are suitable for use in the present invention.

The vaccine preparations of the present invention may be used to protect or treat a mammal susceptible to infection, by means of administering said vaccine via systemic or mucosal route. These administrations may include injection via the intramuscular, intraperitoneal, intradermal or subcutaneous routes; or via mucosal administration to the oral/alimentary, respiratory, genitourinary tracts. Intranasal administration of vaccines for the treatment of pneumonia or otitis media is preferred (as nasopharyngeal carriage of pneumococci can be more effectively prevented, thus attenuating infection at its earliest stage). Although the vaccine of the invention may be administered as a single dose, components thereof may also be co-administered together at the same time or at different times (for instance pneumococcal polysaccharides could be administered separately at the same time or 1-2 weeks after the administration of the bacterial protein component of the vaccine for optimal coordination of the immune responses with respect to each other). For co-administration, the optional Th1 adjuvant may be present in any or all of the different administrations, however it is preferred if it is present in combination with the bacterial protein component of the vaccine. In addition to a single route of administration, 2 different routes of administration may be used. For example, polysaccharides may be administered IM (or ID) and bacterial proteins may be administered IN (or ID). In addition, the vaccines of the invention may be administered IM for priming doses and IM or IN (without aluminum) for booster doses.

The amount of conjugate antigen in each vaccine dose is selected as an amount which induces an immunoprotective response without significant, adverse side effects in typical vaccines. Such amount will vary depending upon which specific immunogen is employed and how it is presented. Generally, it is expected that each dose will comprise 0.1-100 μg of polysaccharide, for polysaccharide conjugates 0.1-50 μg of polysaccharide, preferably 1-10 μg, of which 1 to 5 μg is a preferred range and 2-5 μg is a more preferable range. However, for serotype 6B, the preferred dosage will comprise 3-10 μg of polysaccharide, more preferably 5-10 μg of polysaccharide conjugate.

The content of protein antigens in the vaccine will typically be in the range 1-100 μg, preferably 5-50 μg, most typically in the range 5-25 μg. Following an initial vaccination, subjects may receive one or several booster immunizations adequately spaced.

Vaccine preparation is generally described in Vaccine Design (“The subunit and adjuvant approach” (eds Powell M. F. & Newman M. J.) (1995) Plenuin Press New York). Encapsulation within liposomes is described by Fullerton, U.S. Pat. No. 4,235,877.

The vaccines of the present invention may be stored in solution or lyophilized. As a liquid, the vaccine of the invention is typically stored in 0.5 ml solution/dose. Preferably the vaccine is adsorbed onto an aluminum salt. If the solution is lyophilized, it is preferably in the presence of a sugar such as sucrose or lactose or trelalose. It is still further preferable that they are lyophilized and extemporaneously reconstituted prior to use. Lyophilizing of Streptococcus polysaccharides may result in a more stable composition (vaccine) and may possibly lead to higher antibody titers in the presence of 3D-MPL and in the absence of an aluminum based adjuvant.

Although the vaccines of the present invention may be administered by any route, administration of the described vaccines into the skin (ID) forms one embodiment of the present invention. Human skin comprises an outer “horny” cuticle, called the stratum corneum, which overlays the epidermis. Underneath this epidermis is a layer called the dermis, which in turn overlays the subcutaneous tissue. Researchers have shown that injection of a vaccine into the skin, and in particular the dermis, stimulates an immune response, which may also be associated with a number of additional advantages. Intradermal vaccination with the vaccines described herein forms a preferred feature of the present invention.

The conventional technique of intradermal injection, the “mantoux procedure”, comprises steps of cleaning the skin, and then stretching with one hand, and with the bevel of a narrow gauge needle (26-31 gauge) facing upwards the needle is inserted at an angle of between 10-15°. Once the bevel of the needle is inserted, the barrel of the needle is lowered and further advanced whilst providing a slight pressure to elevate it under the skin. The liquid is then injected very slowly thereby forming a bleb or bump on the skin surface, followed by slow withdrawal of the needle.

More recently, devices that are specifically designed to administer liquid agents into or across the skin have been described, for example the devices described in WO 99/34850 and EP 1092444, also the jet injection devices described for example in WO 01/13977; U.S. Pat. No. 5,480,381, U.S. Pat. No. 5,599,302, U.S. Pat. No. 5,334,144, US 5,993,412, U.S. Pat. No. 5,649,912, U.S. Pat. No. 5,569,189, U.S. Pat. No. 5,704,911, U.S. Pat. No. 5,383,851, U.S. Pat. No. 5,893,397, U.S. Pat. No. 5,466,220, U.S. Pat. No. 5,339,163, U.S. Pat. No. 5,312,335, U.S. Pat. No. 5,503,627, U.S. Pat. No. 5,064,413, U.S. Pat. No. 5,520,639, U.S. Pat. No. 4,596,556, U.S. Pat. No. 4,790,824, U.S. Pat. No. 4,941,880, U.S. Pat. No. 4,940,460, WO 97/37705 and WO 97/13537. Alternative methods of intradermal administration of the vaccine preparations may include conventional syringes and needles, or devices designed for ballistic delivery of solid vaccines (WO 99/27961), or transdermal patches (WO 97/48440. WO 98/28037); or applied to the surface of the skin (transdermal or transcutaneotus delivery WO 98/20734; WO 98/28037).

When the vaccines of the present invention are to be administered to the skin, or more specifically into the dermis, the vaccine is in a low liquid volume, particularly a volume of between about 0.05 ml and 0.2 ml.

The content of antigens in the skin or intradermal vaccines of the present invention may be similar to conventional doses as found in intramuscular vaccines (see above). However, it is a feature of skin or intradermal vaccines that the formulations may be “low dose”. Accordingly the protein antigens in “low dose” vaccines are preferably present in as little as 0.1 to 10 μg, preferably 0.1 to 5 μg per dose; and the polysaccharide (preferably conjugated) antigens may be present in the range of 0.01-1μg, and preferably between 0.01 to 0.5 μg of polysaccharide per dose.

As used herein, the term “intradermal delivery” means delivery of the vaccine to the region of the dermis in the skin. However, the vaccine will not necessarily be located exclusively in the dermis. The dermis is the layer in the skin located between about 1.0 and about 2.0 mm from the surface in human skin, but there is a certain amount of variation between individuals and in different parts of the body. In general, it can be expected to reach the dermis by going 1.5 mm below the surface of the skin. The dermis is located between the stratum corneum and the epidermis at the surface and the subcutaneous layer below. Depending on the mode of delivery, the vaccine may ultimately be located solely or primarily within the dermis, or it may ultimately be distributed within the epidermis and the dermis.

In order that this invention may be better understood, the following examples are set forth. These examples are for purposes of illustration only, and are not to be construed as limiting the scope of the invention in any manner.

### EXAMPLES

### Example 1

Determination of the Polysaccharides to which the Immune Response is Regulated with Age

Human antibody titers to both pre-immune and post-immunization (2 weeks to 3 months) polysaccharides (unconjugated) were collected either internally or via external sources. FIG. 1 shows the relationship between the immunogenicity of each serotype polysaccharide, as measured by the geometric mean fold-increase in antibody titre (GFI) after polysaccharide immunization, and the mean age of the subjects in the study. The linear correlations of log geometric mean fold increase and age give an indication if the immune response is regulated with age. As shown in FIG. 1, serotypes 6, 14, 19 and 23 are significantly correlated with age (p<0.001), whereas serotypes 8, 12 and 18 are less significantly correlated with age (0.05<p<0.2). Finally, serotypes 1, 2, 3, 4, 5, 7 and 9 are not significantly correlated with age (p>or =0.20).

### Example 2

General Methodology of Determining Antibody Responses in Various Mammals

The sera were tested for IgG antibodies to the pneumococcal polysaccharides by an ELISA based on a consensus assay for human sera proposed by the joint CDCAWHO workshops held between 1994 and 1996 (WHO 1996, Plikatis et al J. Clin. Microbiol 38: 2043 (2000)). Briefly, purified capsular polysaccharides from ATCC (Rockville, Md., 20852) were coated at 25 μg/ml in phosphate buffered saline (PBS) on high binding microtitre plates (Nunc Maxisorp) overnight at 4 C. The plates were blocked with 10% fetal calf serum (FCS), 1 hour at 37 C. Serum samples were pre-incubated with the 20 μg/ml cell-wall polysaccharide (Statens Serum Institute, Copenhagen) and 10% FCS at room temperature for 30 minutes to neutralize antibodies to this antigen. A reference serum 89SF (courtesy of Dr. C Frasch, USFDA) was treated in the same fashion, and included on every plate. The samples were then diluted two-fold on the microplate in 10% FCS in PBS, and equilibrated at room temperature for 1 hour with agitation. After washing, the microplates were equilibrated with peroxidase labelled anti-human IgG Fc monoclonal antibody (HP6043-HRP, Stratech Scientific Ltd) diluted 1:4000 in 10% FCS in PBS for 1 hour at room temperature with agitation. The ELISA was performed to measure rat IgG using Jackson ImmunoLaboratories Inc. peroxidase-conjugated AffiniPure Goat anti-Rat IgG (H+L) (code 112-035-003) at 1:5000. The titration curves were referenced to standard sera for each serotype using logistic log comparison by SoftMax Pro. The polysaccharide concentrations used to coat the ELISA plate have been fixed at 10 μg/ml for all serotypes except 6B and 23F, where 20 μg/ml has been used. In addition, 100% fetal calf serum was used as the diluent when testing antisera for serotype 6B, as this serotype was prone to non-specific ELISA responses. Serology for serotype 3 on Rhesus sera used mHSA comix for the coating antigen. Tile color was developed using 4 mg OPD (Sigma) per 10 ml pH 4.5 0.1M citrate buffer with 14 μl H2O2 for 15 minutes in the dark at room temperature. The reaction was stopped with 50 μl HCl, and the optical density was read at 490 nm relative to 650 nm. IgG concentrations were determined by reference of titration points to the calibration curve modeled using a 4-parameter logistic log equation calculated by SoftMax Pro software.

To obtain absolute antibody concentrations in μg/ml, pooled reference antisera were calibrated by two independent methods. For rat antisera, the method of Zollinger and Boslego (1981) was used for 11 serotypes, and for 4 serotypes this was compared with values obtained by immunoprecipitation. Excellent correspondence was found between the two methods. For murine sera, purified monoclonal IgGI antibodies were used, and their active concentrations were confirmed by corollary response (PVW 1999). In this case, reasonable correspondence was found. For Rhesus monkey sera, it was demonstrated that the anti-fgG reagents used react equally with human and Rhesus IgG; thus the calibrated human reference sera 89SF (available from the US FDA) was employed to reference the ELISA.

The ELISA to measure the murine and rat IgG to the pneumococcal polysaccharides was similar with the following exceptions. Locally manufactured polysaccharides were used to coat the ELISA plates at 20 μg/ml in PBS for serotypes 6B and 23F, and 10 μg/ml in PBS for serotypes 14 and 19F. Jackson ImmunoLaboratories Inc. Peroxidase-conjugated AffiniPure Goat Anti-mouse IgG (H+L) and AffiniPure Goat Anti-rat IgG (H+L) were employed to detect bound IgG. HP6043-HRP reacted equally with human and Rhesus purified IgG, and so this reagent was used for Rhesus antiserum, and the reference serum was using 89SF.

The reference serum for human and Rhesus serology was 89SF, kindly provided by Dr. Carl Frasch. The universally accepted weight-based concentration calibration values for the human reference serum 89SF for ]gG, IgA and IgM against 10 pneumococcal serotypes using 2 different methods was published (Salazar et al).

The protein ELISA was performed similarly to the polysaccharide ELISA with the following modifications. The protein was coated overnight at 2.0 μg/ml in PBS. The serum samples were diluted in PBS containing 10% foetal calf serum and 0.1% polyvinyl alcohol. Bound human antibody was detected using Sigina Peroxydase-conjugated goat affinity purified antibody to Human IgG Fc (reference A-2290). To calibrate the protein response in the human and Rhesus monkey serology, Sandoglobulin lot 069, found to contain significant anti-protein D antibody, was used as the reference and given an arbitrary value of 100 ELISA units. For murine and rat serology, the antibody concentrations were quantified by performing corollary response by either direct antigen coating, or by antibody capture.

The sera were also tested for their ability to kill live pneumococci in an in vitro opsonophagocytic assay. The opsonophagocytosis assay was adapted from the published protocol (Romero-Steiner et al. 1997), as well as a detailed protocol provided by Sandy Steiner of the CDC as part of a multi-laboratory study.

Two methods were used. In method A, pneumococcal strains provided by the CDC were replaced by SB production strains were used. Secondly, the HL-60 cells were replaced by freshly purified human neutrophils (PMN). The results are expressed as the serum dilution required for 50% bacterial killing.

In method B, the CDC protocol was followed more closely from a published and detailed standardized protocol provided by the CDC as part of a multi-laboratory study (Romero-Steiner 1997, Romero-Steiner 2000).

Briefly, differentiated HL60 cells were centrifuged at 1000 rpm (300×g) and the culture supernatant was drawn off. The cells were resuspended in the assay buffer consisting of HBSS-BSA. If antibiotics were present in the culture media, this procedure was repeated to ensure complete removal of antibiotics.

Serum samples were pre-diluted in advance for 4 assays to optimize volume measurements. It was demonstrated that samples diluted 1:2 in assay buffer yielded stable opsonic titres for at least 5 days if kept at 4° C. Twenty-five μl of diluted sera was added to 25 μl of assay buffer in a microplate round-bottom well. Twofold serial dilution were performed with 25 μl volume, again to optimize volume measurements.

Baby rabbit complement and pneumococcal cultures were kept at −70° C. until use. A 4:2:1 volume combination of activated HL60 cells, freshly thawed pneumococal culture and freshly thawed baby rabbit complement was mixed with vortexing. Twenty-five μl of this mixture was rapidly distributed to the microplate wells containing diluted sera, yielding a final volume of 50 μl. This gave IE 5 HL60, 150 pneumococcal CFU and 7.1% complement concentration per well in the final mixture, except for serotype 6B in which two modifications were made; the final complement concentration was 12.5% and 5% FCS was included in the assay buffer to equalise growth of pneumococci during incubation. The microplates were incubated for 2 hours at 37° C. with 5% CO2 with shaking at 210 rpm.

After incubation, a viable count was made of the pneumococci from a 20 μl aliquot of the wells. Wells containing only assay buffer with no serum were used as the blank wells to determine the exact number of pneumococci added per well. The mean number of CFU in 8 blank wells on each plate was used for subsequent calculations.

The percent killing was calculated relative to the mean of the blank wells. The titre of a serum sample was determined by the maximum reciprocal dilution of serum able to facilitate greater than 50% killing of the pneulnococci. The values are reported as discontinuous titres of 8, 16 32 etc. Samples for which there was less than 50% killing are reported with a titre <8. Samples in which a prozone effect was observed were repeated, and the second result was taken. If a prozone effect was observed again, the result was considered invalid. This occurs in less than 5% of the samples. Samples which had a titre greater than 1024 were repeated starting at a 1:64 dilution.

### Example 3

Effects of Combination of Pneumococcal PS-PD Conjugates on Immunogenicity in Adult Rats

It has been observed that the combination of vaccines into multivalent formulations can result in the decrease in immunogenicity of one or more components of the vaccine. This has been especially observed for conjugate vaccines, and has been called carrier-induced epitopic suppression. The underlying mechanism for this suppression is not well understood, but it tends to happen at higher dosages of carrier protein.

An 11-valent pneumococcal conjugate vaccine is an example of combination vaccines. Since the combination of each serotype's conjugate will add to the total amount of protein used to immunize, it is important to determine whether the combination of each conjugate vaccine into a multivalent formulation results in a significant decrease in the immunogenicity of the conjugate.

Protocol:

Adult rats were immunized with pneumococcal-polysaccharide protein D conjugate vaccines (see, WO00/56360) either individually, or combined in a multivalent formulation. Groups of 10 rats were immunized twice 28 days apart, and test bleeds were obtained on day 28 and day 42 (14 days after the second dose).

Antibody concentration was measured as described. The opsonic titres were measured according to method A.

Results:

All conjugates induced specific IgG antibodies as measured by ELISA (FIG. 2). Opsonic activity (as determined by the reciprocal of the dilution of pooled sera able to kill 50% of live pneumococci) was also detected in all sera.

FIG. 2 also shows the effect of combination of monovalent PS-PD conjugates on their immunogenicity in adult rats, as measured by IgG concentration and opsonic titre at 14 days post II.

Statistical analysis was performed on all samples to determine if differences in IgG concentration upon combination were significant. Only type 14 showed a significant decrease in ELISA titers upon combination. The IgG concentration was reduced to levels that were similar to the other serotypes. All other differences were not significant, but type 7F approached significance (p=0.08).

Serotypes 1, 3, 6B, 9V and 23F actually show increases upon combination.

### Example 4

Independent Variation of the Dosage of Serotypes 6B and 23F

Combination of individual conjugate vaccines into a multi-valent formulation results in increases or decreases of the antibody response. The immune regulation of the response is serotype dependent. To characterize the immune response to a combined 11-Valent conjugate vaccine, an experiment was undertaken which combined the 11 valences in two groups, 6B and 23F together, versus the remaining 9 valences.

Protocol:

Infant and Adult rats were immunized with 11-Valent PS-PD pneumococcal conjugate vaccine in a two-tiered dosage, that is, the 6B&23F dosage varied independently from the other 9 valence, as shown in Table 1.

TABLE 1
The 11-valent PS-PD Two-Way Dosage Formulation

| | | Dosage 1, 3, 4, 5, |
|---|---|---|
| | Dosage 6B and 23F | 7F, 9V, 14, 18C, |
| Group | (μg) | 19F (μg) |
| 1 | 0.01 | 0.01 |
| 2 | 0.01 | 0.1 |
| 3 | 0.01 | 1 |
| 4 | 0.1 | 0.01 |
| 5 | 0.1 | 0.1 |
| 6 | 0.1 | 1 |
| 7 | 1 | 0.01 |
| 8 | 1 | 0.1 |
| 9 | 1 | 1 |

Infant OFA rats were randomized to different mothers and were 7 days old when they received the first immunization. Ten rats per group received 3 immunizations on days 0, 14 and 28. Bleeds were performed on day 42 (14 days post III) and 56 (28 days post III).

Results:

3D analysis of the two-tiered dosages indicates immune regulation in infant rats caused by 6B-PD and 23F-PD. FIG. 3 shows the GMC for 11 serotypes and PD versus the dosage of 6B and 23F in one dimension, and the dosage of the 9 others in the second dimension. The trend is always the same for all serotypes and PD. Increasing the dosage of 6B and 23F has a dramatic effect on decreasing the antibody response to the remaining conjugates, even though the dosage of those conjugates is unchanged. This effect is very strong in the infant rats, but only slightly observable in the adult rats (not shown).

FIG. 4 shows the antibody concentration against each serotype in the conjugate vaccine as a function of total Protein D content. If carrier-induced epitopic suppression was the principle or only cause of reduction in the immune response with increasing vaccine dosage, it is expected that these curves would be monotonically decreasing. Rather, the wave function indicates there is some other factor influencing the antibody response. As noted from FIG. 3, when the dosage is divided combining serotypes 6B and 23F, a smooth 3D surface is obtained, indicating that 6B and 23F regulate the immune response to the other serotypes. Because in FIG. 4 serotype 6B does show a monotonically decreasing immune response, it may be surmised the dosage of serotype 6B is the dominant factor, as its interaction with itself is always constant, and thus it only shows the effect of carrier-induced epitopic suppression.

Conclusions:

Independent variation of the dosage of 6B&23F and the other 9 serotypes revealed that the dosage of serotypes 6B&23F exerted an influence on the antibody response to the other serotypes. The antibody response to each serotype was reduced with increasing total amount of PD immunized, indicating carrier-induced epitopic suppression, but since the relation is not smooth, there is an additional factor. In addition, the IgG response to PD also decreases with increasing dosage, opposite to what is expected from carrier-induced epitopic suppression. Taken together, these imply a heretofore-unknown regulation of the immune response to conjugate vaccines mapped onto the dosage of serotypes 6B and 23F.

### Example 5

Demonstration that the Immune Regulation from Serotypes 6B and 23F is Transmitted via the Protein Carrier.

Objective:

It is apparent that the dosage of conjugates 6B and 23F regulate the antibody response to the other conjugates in a multivalent formulation. The following experiment was performed to determine if the immune regulation associated with 6B&23F-PD (conjugates) in infant rats was due to the polysaccharide, or the polysaccharide protein conjugate.

Protocol:

Conjugates 6B&23F-PD or PS (unconjugated) were combined with other serotypes in a multivalent formulation, with the dosage of 6B&23F at 0.01 and 1.0 μg, and with the plain polysaccharide at 1.0 μg (without 6B&23F conjugates).

Infant OFA rats were randomized to different mothers and were 7 days old when they received the first immunization. Ten rats per group received 3 immunizations on days 0, 14 and 28. Bleeds were performed on day 42 (14 days post M).

Results:

As previously observed, an increase in the 6B&23F-PD dosage decreased the response to 19F. When PS replaced the conjugate, a higher response to 19F was observed.

Conclusion:

The presence of a 1 μg dosage of 6B and 23F conjugate vaccine is sufficient to regulate the immune response to serotype 19F in a multivalent conjugate vaccine, however, the same dosage of plain polysaccharide has no effect. Since it has been determined that serotypes 6B and 23F are regulated in their immune response in humans and animals, we may conclude the immune regulation of serotypes 6B and 23F are transmitted to the other serotypes via the common protein carrier.

### Example 6

Modification of the Protein Carrier for Serotype 6B

The seroconversion rate of against 6B PS-conjugate was low in the infant rat at 0.1 ug dosage. Other factors that could influence the immunogenicity of the conjugate were examined. These include the ratio of carbohydrate to protein present in the material, the specific method of linkage used, the presence of free polysaccharide, and the specific carrier protein used.

Modification of the coupling chemistry did not increase the immunogenicity of the 6B conjugates in either the infant rat or mouse models. It does appear that the use of the TT carrier increases immunogenicity in the mouse model, but only at a higher dosage. Conjugates were synthesized with an initial carrier protein (protein D)/PS ration of 2.5:1. Other conjugates were synthesized with an initial carrier protein (Protein D)/PS ratio of 1:1.

### Example 7

Clinical Evaluations

Several vaccine formulations of the present invention are undergoing clinical evaluation in humans. Table 2 illustrates the composition of such vaccines.

TABLE 2
S. pneumoniae formulations

| Strep PS serotype: | N^{o}1 | | N^{o}2 | | N^{o}3 | | N^{o}4 | |
|---|---|---|---|---|---|---|---|---|
| 6B (μgPS -carrier) | 10 | μg -DT | 5 | μg -DT | 10 | μg -DT | 5 | μg -DT |
| 19F (μgPS -carrier) | 3 | μg -DT | 3 | μg -DT | 5 | μg -PD | 3 | μg -PD |
| 23F (μgPS -carrier) | 5 | μg -DT | 5 | μg -DT | 5 | μg -DT | 5 | μg -DT |
| 1 (μgPS -carrier) | 3 | μg -PD | 3 | μg -PD | 5 | μg -PD | 3 | μg -PD |
| 3 (μgPS -carrier) | 3 | μg -PD | 2 | μg -PD | 5 | μg -PD | 3 | μg -PD |
| 4 (μgPS -carrier) | 3 | μg -PD | 2 | μg -PD | 5 | μg -PD | 3 | μg -PD |
| 5 (μgPS -carrier) | 3 | μg -PD | 3 | μg -PD | 5 | μg -PD | 3 | μg -PD |
| 7F (μgPS -carrier) | 3 | μg -PD | 3 | μg -PD | 5 | μg -PD | 3 | μg -PD |
| 9V (μgPS -carrier) | 3 | μg -PD | 2 | μg -PD | 5 | μg -PD | 3 | μg -PD |
| 14 (μgPS -carrier) | 3 | μg -PD | 2 | μg -PD | 5 | μg -PD | 3 | μg -PD |
| 18C (μgPS -carrier) | 3 | μg -PD | 2 | μg -PD | 5 | μg -PD | 3 | μg -PD |
| Total PS content: | 42 | μg PS | 32 | μg PS | 60 | μg PS | 37 | μg PS |
| Protein carrier content: | ˜22 | μg PD | ˜18 | μg PD | ˜42 | μg PD | ˜25 | μg PD |
| | ˜18 | μg DT | ˜14 | μg DT | ˜15 | μg DT | ˜10 | μg DT |
| Alum content (Al³⁺): | ˜0.21 | mg | ˜0.16 | mg | ˜0.32 | mg | ˜0.19 | mg |
| +/− Combination with | | | | | | | | |

| | | | | | |
|---|---|---|---|---|---|
| MenC_{ads lyo}: | μg PS | 10 | μg PSC | 10 | μg PSC |
| | μg TT | ˜10 | μg TT | ˜10 | μg TT |
| | μg Al³⁺ | 0.05 | mg Al³⁺ | 0.05 | mg Al³⁺ |

While the preferred embodiments of the invention are illustrated by the above, it is to be understood that the invention is not limited to the precise instructions herein disclosed and that the right to all modifications coming within the scope of the following claims is reserved.

1. An improved Streptococcus pneumoniae vaccine comprising 11 or more polysaccharides from different S. pneumonia serotypes conjugated to 2 or more carrier proteins wherein, serotypes 6B, 19F and 23F are conjugated to a first carrier protein and remaining serotypes are conjugated to 1 or 2 secondary carrier proteins, and wherein the secondary carrier proteins are different from the first carrier protein.

2. An improved Streptococcus pneumoniae vaccine comprising 11 or more polysaccharides from different S. pneumonia serotypes conjugated to 2 or more carrier proteins wherein, serotypes 6B, and 23 F are conjugated to a first carrier protein and remaining serotypes are conjugated to 1 or 2 secondary carrier proteins, and wherein the secondary carrier protein are different from the first carrier protein.

3. An improved Streptococcus pneumoniae vaccine comprising 11 or more polysaccharides from different S. pneumonia serotypes conjugated to 2 or more carrier proteins wherein, serotype 6B is conjugated to a first carrier protein and remaining serotypes are conjugated to 1 or 2 secondary carrier proteins, and wherein the secondary carrier protein are different from the first carrier protein.

4. The vaccine of claim 1 wherein the first carrier protein is selected from the group consisting of DT, crm197, TT, Fragment C, Ply, PhtA, PhtB, PhtD, PhtE, OmpC and PorB.

5. The vaccine of claim 1 wherein the secondary carrier protein comprises one or 2 proteins selected from the group consisting of PD, DT, crm197, TT, Fragment C, Ply, PhtA, PhtB, PhtD, PhtE, OmpC and PorB.

6. The vaccine of claim 1 wherein there is 1 secondary carrier protein.

7. The vaccine of claim 1 wherein polysaccharides of each serotype are present in an amount of 1-10 μg.

8. The vaccine of claim 7 wherein one or more serotypes selected from the group consisting of 1, 3, 4, 5, 7F, 9V, 14 and 18C are present in an amount of 2-5 μg.

9. The vaccine of claim 1 wherein the ratio of carrier protein to polysaccharide is 0.5 to 1.7 (w/w).

10. The vaccine of claim 9 wherein the ratio of carrier protein to polysaccharide is from 0.7 to 1.5 for one or more serotypes selected from the group consisting of 6B, 19F and 23F.

11. The vaccine of claim 1 wherein the secondary carrier protein is H. influenzae protein D (PD).

12. The vaccine of claim 1 wherein polysaccharide serotype 6B is conjugated to a first carrier protein selected from the group consisting of DT, crm197 or TT.

13. The vaccine of claim 12 wherein the first carrier protein is DT.

14. The vaccine of claim 1 wherein polysaccharide 6B is present in an amount of 5-10 μg/dose.

15. The vaccine of claim 1 further comprising unconjugated S. pneumoniae polysaccharides of serotypes different from those conjugated, such that the number of conjugated and unconjugated polysaccharides is less than or equal to 23.

16. A method of eliciting a protective immune response to infants 0-2 years old against S. pneumoniae by administering the vaccine of claim 1.

17. A method of eliciting a protective immune response to the elderly aged 50 years or over against S. pneumoniae by administering the (i) vaccine of claim 1 and (ii) a S. pneumoniae surface protein from the PhtX family, wherein the S. pneumoniae surface protein is different from the first and secondary carrier proteins.

18. A method of eliciting a protective immune response to infants 0-2 years old against Otitis media by administering the (i) vaccine of claim 1 and (ii) a S. pneumoniae surface protein from the PhtX family, wherein the S. pneumoniae surface protein is different from the first and secondary carrier proteins.

19. The method of claim 17 wherein the PhtX family protein is PhtD or PhtB.

20. The method of claim 19 wherein the PhtX family protein is PhtD.

21. The method of claim 17 further comprising a CbpX family protein.

22. The method of claim 21 wherein the CbpX protein is a truncate lacking the choline binding domain.

23. The method of claim 22 wherein the CbpX truncate is choline binding protein A.

24. The method of claim 17 further comprising Ply.

25. The vaccine of claim 2 wherein the first carrier protein is selected from the group consisting of DT, crm197, TT, Fragment C, Ply, PhtA, PhtB, PhtD, PhtE, OmpC and PorB.

26. The vaccine of claim 2 wherein the secondary carrier protein comprises one or 2 proteins selected from the group consisting of PD, DT, crm197, TT, Fragment C, Ply, PhtA, PhtB, PhtD, PhtE, OmpC and PorB.

27. The vaccine of claim 2 wherein there is 1 secondary carrier protein.

28. The vaccine of claim 2 wherein polysaccharides of each serotype are present in an amount of 1-10 μg.

29. The vaccine of claim 28 wherein one or more serotypes selected from the group consisting of 1, 3, 4, 5, 7F, 9V, 14 and 18C are present in an amount of 2-5 μg.

30. The vaccine of claim 2 wherein the ratio of carrier protein to polysaccharide is 0.5 to 1.7 (w/w).

31. The vaccine of claim 30 wherein the ratio of carrier protein to polysaccharide is from 0.7 to 1.5 for one or more serotypes selected from the group consisting of 6B, 19F and 23F.

32. The vaccine of claim 2 wherein the secondary carrier protein is H. influenzae protein D (PD).

33. The vaccine of claim 2 wherein polysaccharide serotype 6B is conjugated to a first carrier protein selected from the group consisting of DT, crm197 or TT.

34. The vaccine of claim 33 wherein the first carrier protein is DT.

35. The vaccine of claim 2 wherein polysaccharide 6B is present in an amount of 5-10 μg/dose.

36. The vaccine of claim 2 further comprising unconjugated S. pneumoniae polysaccharides of serotypes different from those conjugated, such that the number of conjugated and unconjugated polysaccharides is less than or equal to 23.

37. A method of eliciting a protective immune response to infants 0-2 years old against S. pneumoniae by administering the vaccine of claim 2.

38. A method of eliciting a protective immune response to the elderly aged 50 years or over against S. pneumoniae by administering the (i) vaccine of claim 2 and (ii) a S. pneumoniae surface protein from the PhtX family, wherein the S. pneumoniae surface protein is different from the first and secondary carrier proteins.

39. A method of eliciting a protective immune response to infants 0-2 years old against Otitis media by administering the (i) vaccine of claim 2 and (ii) a S. pneumoniae surface protein from the PhtX family, wherein the S. pneumoniae surface protein is different from the first and secondary carrier proteins.

40. The method of claim 38 wherein the PhtX family protein is PhtD or PhtB.

41. The method of claim 40 wherein the PhtX family protein is PhtD.

42. The method of claim 38 further comprising a CbpX family protein.

43. The method of claim 42 wherein the CbpX protein is a truncate lacking the choline binding domain.

44. The method of claim 43 wherein the CbpX truncate is choline binding protein A.

45. The method of claim 38 further comprising Ply.

46. The vaccine of claim 3 wherein the first carrier protein is selected from the group consisting of DT, crm197, TT, Fragment C, Ply, PhtA, PhtB, PhtD, PhtE, OmpC and PorB.

47. The vaccine of claim 3 wherein the secondary carrier protein comprises one or 2 proteins selected from the group consisting of PD, DT, crm197, TT, Fragment C, Ply, PhtA, PhtB, PhtD, PhtE, OmpC and PorB.

48. The vaccine of claim 3 wherein there is 1 secondary carrier protein.

49. The vaccine of claim 3 wherein polysaccharides of each serotype are present in an amount of 1-10 μg.

50. The vaccine of claim 49 wherein one or more serotypes selected from the group consisting of 1, 3, 4, 5, 7F, 9V, 14 and 18C are present in an amount of 2-5 μg.

51. The vaccine of claim 3 wherein the ratio of carrier protein to polysaccharide is 0.5 to 1.7 (w/w).

52. The vaccine of claim 51 wherein the ratio of carrier protein to polysaccharide is from 0.7 to 1.5 for one or more serotypes selected from the group consisting of 6B, 19F and 23F.

53. The vaccine of claim 3 wherein the secondary carrier protein is H. influenzae protein D (PD).

54. The vaccine of claim 3 wherein polysaccharide serotype 6B is conjugated to a first carrier protein selected from the group consisting of DT, crm197 or TT.

55. The vaccine of claim 54 wherein the first carrier protein is DT.

56. The vaccine of claim 3 wherein polysaccharide 6B is present in an amount of 5-10 μg/dose.

57. The vaccine of claim 3 further comprising unconjugated S. pneumoniae polysaccharides of serotypes different from those conjugated, such that the number of conjugated and unconjugated polysaccharides is less than or equal to 23.

58. A method of eliciting a protective immune response to infants 0-2 years old against S. pneumoniae by administering the vaccine of claim 3.

59. A method of eliciting a protective immune response to the elderly aged 50 years or over against S. pneumoniae by administering the (i) vaccine of claim 3 and (ii) a S. pneumoniae surface protein from the PhtX family, wherein the S. pneumoniae surface protein is different from the first and secondary carrier proteins.

60. A method of eliciting a protective immune response to infants 0-2 years old against Otitis media by administering the (i) vaccine of claim 3 and (ii) a S. pneumoniae surface protein from the PhtX family, wherein the S. pneumoniae surface protein is different from the first and secondary carrier proteins.

61. The method of claim 59 wherein the PhtX family protein is PhtD or PhtB.

62. The method of claim 61 wherein the PhtX family protein is PhtD.

63. The method of claim 59 further comprising a CbpX family protein.

64. The method of claim 63 wherein the CbpX protein is a truncate lacking the choline binding domain.

65. The method of claim 64 wherein the CbpX truncate is choline binding protein A.

66. The method of claim 59 further comprising Ply.


SEQUENCE LISTING (2 sequences)

SEQ ID NO: 1 - protein, 4 aa, Streptococcus pneumoniae
  VARIANT (1)...(4): Xaa = Any Amino Acid
LXXC

SEQ ID NO: 2 - protein, 5 aa, Streptococcus pneumoniae
  VARIANT (1)...(5): Xaa = Any Amino Acid
LPXTG